(19) United States
(12) Patent Application Publication
(10) Pub. No.: US 2021/0079414 A1
(43) Pub. Date: Mar. 18, 2021

(54) USE OF PROTEIN NOG1 IN REGULATION OF PLANT YIELD AND GRAIN NUMBER PER EAR

(71) Applicant: China Agricultural University, Beijing (CN)

(72) Inventors: Chuanqing Sun, Beijing (CN); Xing Huo, Beijing (CN); Lubin Tan, Beijing (CN); Fengxia Liu, Beijing (CN); Yongcai Fu, Beijing (CN); Zuofeng Zhu, Beijing (CN); Ping Gu, Beijing (CN)

(21) Appl. No.: 16/603,199

(22) PCT Filed: Aug. 16, 2017
(86) PCT No.: PCT/CN2017/097608

§ 371 Date: Oct. 4, 2019

(30) Foreign Application Priority Data
Apr. 6, 2017 (CN) 201710220258.6

(51) Int. Cl. C12N 15/82

(52) U.S. Cl. CPC C12N 15/8261; C12N 15/8218

(57) ABSTRACT

Protein nog1 is used in the regulation of plant yield and/or grain number. The transgenic treated Guichao 2 in which the expression of the protein nog1 is inhibited has a reduced yield per plant and/or a reduced grain number of the main stem as compared with untreated Guichao 2. The transgenic plant obtained by introducing a nucleic acid molecule encoding the protein nog1 into SIL176 has an increased yield per plant and/or an increased grain number of the main stem as compared with untransformed SIL176.

### CROSS-REFERENCED TO RELATED APPLICATIONS

This application is the U.S. National Phase under 35 U.S.C. § 371 of International Application No. PCT/CN2017/097608, filed Aug. 16, 2017, designating the U.S., and published in Chinese as WO 2018/184333 A1 on Oct. 11, 2018, which claims priority to Chinese Patent Application No. 201710220258.6, filed Apr. 6, 2017, the entire contents of which are incorporated herein by reference.

### REFERENCE TO SEQUENCE LISTING

A Sequence Listing submitted as an ASCII text file via EFS-Web is hereby incorporated by reference in accordance with 35 U.S.C. § 1.52(e). The name of the ASCII text file for the Sequence Listing is 31422987_1. TXT, the date of creation of the ASCII text file is Sep. 30, 2019, and the size of the ASCII text file is 13.6 KB.

### TECHNICAL FIELD

The present invention relates to the field of biotechnology, specifically to the use of protein nog1 in the regulation of plant yield and grain number.

### BACKGROUND

Rice, one of the most important food crops in the world, is grown in more than 120 countries around the world, with the cultivated area being maintained at above 150 million hectares per year, and feeds 50% of the population in the world as a staple food. Today, as the population continues to increase and the area of cultivated arable land decreases year by year, increasing rice yield per unit area is one of powerful measures to ensure world food security. Reviewing the history of rice breeding over half a century, the rice yield per unit area of China has experienced two leaps, the first one is the green revolution marked by dwarf breeding, and the second one is the utilization of heterosis of rice. But the rice yield per unit area has been stagnant in the last 20 years. The researchers believe that many of the cultivars currently used in production have the same or similar genetic sources, the utilization of genetic resources of rice cultivars tends to be saturated, and the narrow genetic diversity between rice cultivars results in the similar genetic basis and genotype thereof, which have been a bottleneck restricting the further improvement of potential rice yield.

Common wild rice (Oryza rufipogon Griff.) is a wild ancestor of Asian cultivated rice, and has richer genetic diversity and genetic resources than the artificially domesticated cultivated rice. The common wild rice has far more genetic differentiation types than cultivated rice, and contains abundant genes that can increase rice yield. Therefore, it is of great theoretical significance and practical value to excavate and utilize the excellent domesticated genes that have been lost or weakened in cultivated rice from the genome of common wild rice and apply them to breeding production of rice, which is also an effective way to solve the breeding problem of rice in the current.

### SUMMARY

The technical problem to be solved by the present invention is how to regulate plant yield and grain number.

To solve the above technical problem, the present invention first provides a use of a protein nog1 in the regulation of plant yield and/or grain number; and the protein nog1 can be a1) or a2) or a3) or a4):

a1) a protein with an amino acid sequence as shown in Sequence 2 in the Sequence Listing;

a2) a fusion protein obtained by linking a tag to the N-terminus or/and C-terminus of the protein shown in Sequence 2 in the Sequence Listing;

a3) a protein which relates to plant yield and/or grain number obtained by substitution and/or deletion and/or addition of one or more amino acid residues in the amino acid sequence shown in Sequence 2 in the Sequence Listing; and

a4) a protein having 80% or more identity with the amino acid sequence defined in a1).

Wherein, Sequence 2 in the Sequence Listing consists of 389 amino acid residues.

To facilitate the purification of the protein of a1), a tag as shown in Table 1 may be linked to the amino terminus or the carboxyl terminus of the protein shown in Sequence 2 in the Sequence Listing.

TABLE 1
Sequence of the tag

| Tag | Residues | Sequence |
|---|---|---|
| Poly-Arg | 5-6 (usually 5) | RRRRR |
| FLAG | 8 | DYKDDDDK |
| Strep-tag II | 8 | WSHPQFEK |
| c-myc | 10 | EQKLISEED |
| | | L |

The substitution and/or deletion and/or addition of one or more amino acid residues in the protein in above a3) is a substitution and/or deletion and/or addition of no more than 10 amino acid residues.

The protein in above a3) can be artificially synthesized, and also can be obtained by synthesizing the encoding gene thereof firstly and then performing biological expression.

The encoding gene of the protein in above a3) can be obtained by deleting codon (s) of one or more amino acid residues in the DNA sequence shown in Sequence 1 in the Sequence Listing, and/or performing the missense mutations of one or more base pairs, and/or linking the tag shown in Table 1 to the encoding gene at its 5′ end and/or 3′ end.

The term “identity” as used in above a4) refers to the sequence similarity to the amino acid sequence of the protein shown in Sequence 2 in the Sequence Listing. “Identity” comprises amino acid sequences having 80% or more, or 85% or more, or 90% or more, or 95% or more identity with the amino acid sequence shown in Sequence 2 in the Sequence Listing of the present invention. Identity can be evaluated with naked eyes or computer software. Using the computer software, the identity between two or more sequences can be expressed in percentage (%), which can be used to evaluate the identity between related sequences.

A use of a nucleic acid molecule encoding the protein nog1 in the regulation of plant yield and/or grain number also belongs to the protection scope of the present invention.

The nucleic acid molecule encoding the protein nog1 can be a DNA molecule shown in b1) or b2) or b3) or b4) as below:

b1) a DNA molecule with an encoding region as shown in Sequence 1 in the Sequence Listing;

b2) a DNA molecule with a nucleotide sequence as shown in Sequence 1 in the Sequence Listing;

b3) a DNA molecule having 75% or more identity with the nucleotide sequence defined in b1) or b2) and encoding the protein nog1; and

b4) a DNA molecule which hybridizes to the nucleotide sequence defined in b1) or b2) under stringent conditions and encodes the protein nog1.

Wherein, the nucleic acid molecule may be DNA, such as cDNA, genomic DNA or recombinant DNA; and the nucleic acid molecule may also be RNA, such as mRNA or hnRNA, and the like.

Wherein, Sequence 1 in the Sequence Listing is composed of 1170 nucleotides, and the nucleotides in Sequence 1 in the Sequence Listing encode the amino acid sequence as shown in Sequence 2 in the Sequence Listing.

The nucleotide sequence encoding the protein nog1 of the present invention may be readily mutated by the skilled in the art using known methods, such as directed evolution and point mutation. Those artificially modified nucleotides having 75% or more identity with the nucleotide sequence of the protein nog1 isolated in the present invention, as long as they encode the protein nog1, are all derived from the nucleotide sequence of the present invention and equivalent to the sequence of the present invention.

The term “identity” as used herein refers to the sequence similarity to a native nucleic acid sequence. “Identity” comprises nucleotide sequences having 75% or more, or 80% or more, or 85% or more, or 90% or more, or 95% or more identity with the nucleotide sequences encoding the protein nog1 composed of amino acid sequences shown in Sequence 2 in the Sequence Listing of the present invention. Identity can be evaluated with naked eyes or computer software. Using the computer software, the identity between two or more sequences can be expressed in percentage (%), which can be used to evaluate the identity between related sequences.

In the above use, the regulation of plant yield may be a regulation of plant yield per plant. The regulation of grain number of plant may be a regulation of grain number of the main stem and/or average grain number of the plant.

In the above use, the plant may be any of the following c1) to c7): c1) dicotyledon; c2) monocotyledon; c3) gramineous plant; c4) rice; c5) indica rice; c6) rice variety Guichao 2; and c7) Dongxiang common wild rice introgression line SIL176.

To solve the above technical problem, the present invention further provides a method 1 for cultivating a transgenic plant A or a method 2 for cultivating a transgenic plant B.

The method 1 for cultivating a transgenic plant A provided by the present invention may comprise the step of introducing a nucleic acid molecule encoding the protein nog1 into a recipient plant A to obtain a transgenic plant A; and the transgenic plant A has an increased yield and/or an increased grain number as compared with the recipient plant A.

In the above method 1, said “introducing a nucleic acid molecule encoding the protein nog1 into a recipient plant A” can be achieved by introducing a recombinant vector A into the recipient plant A; and the recombinant vector A can be a recombinant plasmid obtained by inserting a nucleic acid molecule encoding the protein nog1 into an expression vector.

The recombinant vector A may specifically be the recombinant plasmid pCAMBIA1300-NOGE The recombinant plasmid pCAMBIA1300-NOG1 may specifically be a modified plant expression vector pCAMBIA1300 in which the small DNA fragment between the recognition sequence of restriction endonuclease BglII and the recognition sequence of restriction endonuclease MluI is replaced with a DNA molecule with the nucleotide sequence as shown in Sequence 3 in the Sequence Listing.

In the above method 1, the recipient plant A may be any of the following d1)-d6): d1) monocotyledon; d2) dicotyledon; d3) gramineous plant; d4) rice; d5) indica rice; d6) Dongxiang common wild rice introgression line SIL176.

The method 2 for cultivating the transgenic plant A provided by the present invention may comprise the step of introducing a substance which inhibits the expression of the protein nog1 into a recipient plant B to obtain a transgenic plant B; and the transgenic plant B has a reduced yield and/or a reduced grain number as compared with the recipient plant B,

In the above method 2, said “substance which inhibits the expression of the protein nog1” may be a specific DNA molecule, an expression cassette containing the specific DNA molecule, or a recombinant plasmid containing the specific DNA molecule;

The specific DNA molecule comprises a sense fragment, an antisense fragment and a spacer fragment located therebetween; the sense fragment is a reverse complement of the DNA molecule shown at positions 155^{th} to 522^{nd} from the 5′ end of Sequence 1 in the Sequence Listing; and the antisense fragment is a DNA molecule shown at positions 161^{st} to 522^{nd} from the 5′ end of Sequence 1 in the Sequence Listing.

In the above method 2, the recombinant plasmid containing the specific DNA molecule may specifically be a recombinant plasmid pRNAi-nog1. The recombinant plasmid pRNAi-nog1 may specifically be a vector pTCK303/JL1460 in which the small DNA fragment between the recognition sequence of BamHI and the recognition sequence of KpnI is replaced with the reverse complement of the DNA molecule with the nucleotide sequence as shown at positions 155^{th} to 522^{nd} from the 5′ end of Sequence 1 in the Sequence Listing, and the small DNA fragment between the recognition sequence of SpeI and the recognition sequence of SacI is replaced with the DNA molecule with the nucleotide sequence as shown at positions 161^{st} to 522^{nd} from the 5′ end of Sequence 1 in the Sequence Listing.

In the above method 2, the recipient plant B may be any of the following e1)-e6): e1) monocotyledon; e2) dicotyledon; e3) gramineous plant; e4) rice; e5) indica rice; and e6) rice variety Guichao 2.

To solve the above technical problem, the present invention further provides a method 3 for cultivating a transgenic plant C.

The method 3 for cultivating the transgenic plant C provided by the present invention may comprise the step of introducing a substance which increases the expression and/or activity of the protein nog1 into a recipient plant C to obtain a transgenic plant C; and the transgenic plant C has an increased yield and/or an increased grain number as compared with the recipient plant C.

In the above method 3, said “substance which increases the expression and/or activity of the protein nog1” may specifically be the recombinant vector A.

In the above method 3, the recipient plant C may be any of the following d1)-d6): d1) monocotyledon; d2) dicotyledon; d3) gramineous plant; d4) rice; d5) indica rice; and d6) Dongxiang common wild rice introgression line S1L176.

In the above method, the nucleic acid molecule encoding the protein nog1 may be a DNA molecule as shown in b1) or b2) or b3) or b4) below:

b1) a DNA molecule with an encoding region as shown in Sequence 1 in the Sequence Listing;

b2) a DNA molecule with a nucleotide sequence as shown in Sequence 1 in the Sequence Listing;

b3) a DNA molecule having 75% or more identity with the nucleotide sequence defined in b1) or b2) and encoding the protein nog1;

b4) a DNA molecule hybridizing to the nucleotide sequence defined in b1) or b2) under stringent conditions and encoding the protein nog1.

Wherein, the nucleic acid molecule may be DNA, such as cDNA, genomic DNA or recombinant DNA; and the nucleic acid molecule may also be RNA, such as mRNA or hnRNA, and the like.

Wherein, Sequence 1 in the Sequence Listing is composed of 1170 nucleotides, and the nucleotides in Sequence 1 in the Sequence Listing encode the amino acid sequence as shown in Sequence 2 in the Sequence Listing.

To solve the above technical problem, the present invention also provides a method 1 for plant breeding or a method 2 for plant breeding.

The method 1 for plant breeding provided by the present invention may comprise the step of increasing the amount and/or activity of the protein nog1 in a plant, thereby increasing plant yield and/or grain number.

In the above method 1 for plant breeding, the “increasing the amount and/or activity of the protein nog1 in a plant” can achieve the effect of increasing the amount and/or activity of the protein nog1 in the plant by methods well known in the art, such as multi-copy, changing the promoter, regulatory factor and transgene, and the like.

The method 2 for plant breeding provided by the present invention may comprise the step of reducing the amount and/or activity of the protein nog1 in a plant, thereby reducing plant yield and/or grain number.

In the above method 2 for plant breeding, the “reducing the amount and/or activity of the protein nog1 in a plant” can achieve the purpose of reducing the amount and/or activity of the protein nog1 in the plant by methods well known in the art, such as RNA interference, homologous recombination, gene editing, and the like.

In the above methods, the plant can be any of the following f1)-f4): f1) monocotyledon; f2) dicotyledon; f3) gramineous plant; and f4) rice.

In any of the above methods, the yield can be the yield per plant. The grain number can be grain number of the main stem and/or average grain number.

Said “substance which inhibits the expression of the protein nog1” also belongs to the protection scope of the present invention.

Any of the above substances which inhibit the expression of the protein nog1 can specifically be a specific DNA molecule, an expression cassette containing the specific DNA molecule, or a recombinant plasmid containing the specific DNA molecule.

The specific DNA molecule comprises a sense fragment, an antisense fragment and a spacer fragment located therebetween.

The sense fragment is a reverse complement of the DNA molecule shown at positions 155^{th} to 522^{nd} from the 5′ end of Sequence 1 in the Sequence Listing; and the antisense fragment is the DNA molecule shown at positions 161^{st} to 522^{nd} from the 5′ end of Sequence 1 in the Sequence Listing.

A recombinant plasmid containing the specific DNA molecule may specifically be the recombinant plasmid pRNAi-nog1. The recombinant plasmid pRNAi-nog1 may specifically be a vector pTCK303/JL1460 in which the small DNA fragment between the recognition sequence of BamHI and the recognition sequence KpnI is replaced with the reverse complement of the DNA molecule with nucleotide sequence as shown at positions 155^{th} to 522^{nd} from 5′ end of Sequence 1 in the Sequence Listing, and the small DNA molecule between the recognition sequence of SpeI and the recognition sequence of SacI is replaced with the DNA molecule with nucleotide sequence shown at positions 161″ to 522^{nd} from 5′ end of Sequence 1 in the Sequence Listing.

It is demonstrated by the experiments that a transgenic plant B is obtained by introducing a substance which inhibits the expression of protein nog1 (i.e. recombinant plasmid pRNAi-nog1) into Guichao 2; and the transgenic plant B has a reduced yield per plant and/or a reduced grain number of main stem and/or a reduced average grain number as compared with Guichao 2. A transgenic plant A is obtained by introducing the nucleic acid molecule encoding the protein nog1 into SIL176; and the transgenic plant A has an increased yield per plant and/or an increased grain number of main stem and/or an increased average grain number as compared with SIL176.

The results demonstrated that protein nog1 plays an important role in the regulation of yield and grain number of rice.

### BRIEF DESCRIPTION OF THE DRAWINGS

FIG. 1 is a comparison of the (A) morphology of grains of the main stem, (B) grain number of the main stem and (C) yield per plant between SIL176 and Guichao 2.

FIG. 2 is the comparison of the (A) morphology of grains of the main stem, (B) the expression of nog1 gene, (C) grain number of the main stem and (D) yield per plant between homozygous RNAi interference line of T₂ generation and Guichao 2.

FIG. 3 is the comparison of the (A) morphology of grains of the main stem, (B) the expression of nog1 gene, (C) grain number of the main stem and (D) yield per plant between homozygous complemented line of T₂ generation and SIL176.

### CERTAIN EMBODIMENTS

Hereinafter, the present invention will be further described in more detail with reference to the specific embodiments, the given Examples are only illustrative of the present invention, and the scope of the present invention is not limited to these Examples.

The experimental methods in the following examples are conventional methods unless otherwise specified.

The materials, reagents and the like used in the following examples are commercially available unless otherwise specified.

For the quantitative tests in the following examples, three replicate experiments are set, and the results are averaged.

The vector pTCK303/JL1460 is described in the following literature: Wang Z, Chen C G, Xu Y Y, Jiang R X, Han Y, Xu Z H and Chong K. A Practical Vector for Efficient Knockdown of Gene Expression in Rice (Oryza sativa L.). Plant Molecular Biology Reporter, 2004, 22: 409-417.

Guichao 2 is described in the following literature: Zhang X, Zhou S X, Fu Y C, et al. Identification of a drought tolerant introgression line derived from Dongxiang common wild rice (O. rufipogon Griff.). Plant Mol Biol, 2006, 62:247˜259, which is accessible by the public from China Agriculture University. It is hereinafter referred to as Guichao 2. Guichao 2 belongs to indica rice.

Jiangxi Dongxiang wild rice is described in the following literature: Tian F, Li D J, Fu Q, Zhu Z F, Fu Y C, Wang X K, Sun C Q.2006. Construction of introgression lines carrying wild rice (Oryza rufipogon Griff) segments in cultivated rice (O. sativa L.) background and characterization of introgressed segments associated with yield-related traits. Theoretical and Applied Genetics, 112, 570-80. It is accessible by the public from China Agriculture University.

Agrobacterium tumefaciens strain EHA105 (named Agrobacterium tumefaciens strain EHA105 in the literature) is described in the following literature: GLUTELIN PRECURSOR ACCUMULATION3 encodes a regulator of post-Golgi vesicular traffic essential for vacuolar protein sorting in rice endosperm. Plant Ce11.2014 January; 26(1): 410-25. The public can access it from China Agriculture University to repeat the experiments of the present application.

Dongxiang common wild rice introgression line SIL176 is the progeny from multiple crosses and backcrosses between Guichao 2 and Jiangxi Dongxiang wild rice, it is described in the following literature: Tian F, Li D J, Fu Q, Zhu Z F, Fu Y C, Wang X K, Sun C Q. 2006. Construction of introgression lines carrying wild rice (Oryza rufipogon Griff) segments in cultivated rice (O. sativa L.) background and characterization of introgressed segments associated with yield-related traits. Theoretical and Applied Genetics, 112, 570-80. It is accessible by the public from China Agriculture University. Dongxiang common wild rice introgression line is hereinafter referred to as SIL176.

cDNA of Guichao 2: it is obtained by first extracting total RNA with TRIZOL reagent using two-week seedlings of Guichao 2 as experimental materials, and then reverse transcribing with SuperScript II reverse transcriptase. The amount of DNA in cDNA of Guichao 2 is about 200 ng/μL. SuperScript II reverse transcriptase is a product of Invitrogen with a catalog number 18064-014.

The modified plant expression vector pCAMBIA1300: it is a vector obtained by in vector pCAMBIA1300, adding the recognition sequence of restriction endonuclease BglII into the 5′ end of the recognition sequence of the restriction endonuclease KpnI, and adding the recognition sequence of restriction endonuclease MluI into the 5′ end of the recognition sequence of restriction endonuclease BamHI, and remaining the other nucleotide sequences unchanged.

### Example 1. Discovery of nog1 Gene

A set of introgression line group containing 265 lines (one of which is SIL176) is constructed by using Jiangxi Dongxiang wild rice as the donor parent and conducting hybridization and backcrossing with Guichao 2 as the recurrent parent. The coverage of genomes of wild rice in this group reached up to 79.4%, in which 15 lines (one of which is SIL176) have a reduced yield per plant of above 35% as compared with Guichao 2. The morphology of grains of the main stem, grain number of the main stem and yield per plant between Guichao 2 and SIL176 are compared and counted. The experiment is repeated three times, with 30 plants repeated each time.

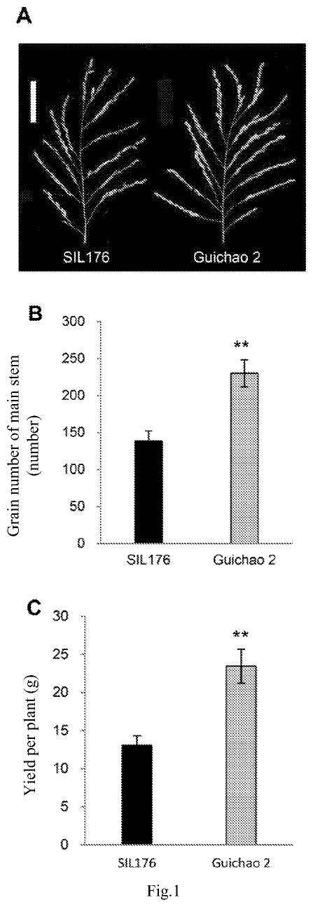

The experimental results are shown in FIG. 1 (A is the morphology of grains of the main stem, bar=5 cm; B is the grain number of the main stem; and C is the yield per plant; ** indicates P<0.01, the difference is extremely significant). The results show that SIL176 has a significantly reduced grain number of the main stem and a significantly reduced yield per plant as compared with Guichao 2.

Map-based cloning and functional analysis are performed on SIL176. As a result, a QTL relating to rice yield is found on the long arm of chromosome 1, and it is named nog1 gene. The Open Reading Frame of nog1 gene is shown in Sequence 1 in the Sequence Listing, the encoded protein is named nog1, the amino acid sequence thereof is shown in Sequence 2 in the Sequence Listing, and consists of 389 amino acid residues.

### Example 2. Obtainment and Phenotypic Identification of Homozygous RNAi Interference Lines of T₂ Generation

I. Construction of Recombinant Plasmid pRNAi-Nog1

The steps of construction of recombinant plasmid pRNAi-nog1 are as follows:

1. Synthesis of the Primers

Primers 860-rnai-320F, 860-rnai-681R, 860-rnai-681F and 860-rnai-314R are designed and synthesized according to the sequence of the nog1 gene as shown in Sequence 1 in the Sequence Listing; and the specific sequences of the primers are as follows:

860-rnai-320F: 5′-GGACTAGTGGGAGAAAGATGAGGA-3′ (the
recognition site of restriction endonuclease
SpeI is underlined);
860-rnai-681R: 5′-TCCGAGCTCGGTCAAAGCCAGGTAC-3′
(the recognition site of restriction endonuclease
SacI is underlined);
860-rnai-681F: 5′-CGGGATCCGGTCAAAGCCAGGTAC-3′ (the
recognition site of restriction endonuclease BamHI
is underlined);
860-rnai-314R: 5′-GGGGTACCAGAGCTGGGAGAAAGA-3′ (the
recognition site of restriction endonuclease KpnI
is underlined).

2. PCR amplification is performed using the cDNA of Guichao 2 as a template and 860-rnai-320F and 860-rnai-681R as the primers, to obtain a DNA fragment A of about 360 bp.

3. PCR amplification is performed using the cDNA of Guichao 2 as a template and 860-rnai-681F and 860-rnai-314R as the primers, to obtain a DNA fragment B of about 360 bp.

4. The DNA fragment A is digested with restriction endonucleases SpeI and SacI, and a digested product 1 is recovered.

5. The vector pTCK303/JL1460 is digested with restriction endonucleases SpeI and SacI, and a vector backbone 1 of about 14.6 kb is recovered.

6. The digested product 1 is ligated to the vector backbone 1 to obtain an intermediate plasmid.

7. The DNA fragment B is digested with restriction endonucleases BamHI and KpnI, and a digested product 2 is recovered.

8. The intermediate plasmid is digested with restriction endonucleases BamHI and KpnI, and a vector backbone 2 of about 14.9 kb is recovered.

9. The digested product 2 is ligated to the vector backbone 2 to obtain a recombinant plasmid pRNAi-nog1.

According to the sequencing results, the recombinant plasmid pRNAi-noga is structurally described as follows: the vector pTCK303/JL1460 in which the small DNA fragment between the recognition sequence of BamHI and the recognition sequence of KpnI is replaced with the reverse complement of a DNA molecule with the nucleotide sequence as shown at positions 155^{th} to 522^{nd} from 5′ end of Sequence 1 in the Sequence Listing, and the small DNA fragment between the recognition sequence of SpeI and the recognition sequence of SacI is replaced with the DNA molecule with the nucleotide sequence as shown at positions 161″ to 522^{nd} from the 5′ end of Sequence 1 in the Sequence Listing.

II. Obtainment of Agrobacterium

The recombinant plasmid pRNAi-nog1 is introduced into Agrobacterium tumefaciens

EHA105 to obtain a recombinant Agrobacterium EHA105/pRNAi-nog1.

III. Obtainment of RNAi interference plant of T₀ generation

The recombinant Agrobacterium EHA105/pRNAi-nog1 is transformed into Guichao 2 using the method of Hiei et. al. (Hiei Y, Ohta S, Komari T & Kumashiro T. Efficient transformation of rice (Oryza sativa L.) mediated by Agrobacterium and sequence analysis of the boundaries of the T-DNA. Plant J. 1994, 6:271-282) to obtain RNAi interference plants of T₀ generation.

IV. Real-Time Quantitative PCR Detection of RNAi Interference Plants of T₀ Generation

Three RNAi interference plants of T₀ generation (named as RNAi-1-T₀ to RNAi-3-T₀ respectively) are randomly selected for real-time quantitative PCR detection, the specific steps are as follows:

1. Total RNA is first extracted with TRIZOL reagent using two-week seedlings of three RNAi interference plants of T₀ generation as experimental materials, respectively, and then reverse transcribing is performed using SuperScriptII reverse transcriptase to obtain cDNAs of each plant of T₀ generation to be silenced. The amount of DNA in the cDNA of each of three RNAi interference plants of T₀ generation is about 200 ng/μL.

2. The relative expression of nog1 gene in three RNAi interference plants of T₀ generation is respectively detected using RT-qPCR technique (using UBI gene as internal reference gene).

The primers for detecting nog1 gene are forward primer 1: 5′-TCCGACTTACAATGAACAC-3′ and reverse primer 1: 5′-GGTAGCAGGACTCCACTT-3′. The primers for detecting UBI gene are forward primer 2: 5′-CTGTCAACTGCCGCAAGAAG-3′ and reverse primer 2: 5′-GGCGAGTGACGCTCTAGTTC-3′.

According to the above method, the RNAi interference plant of T₀ generation is replaced with Guichao 2, and the other steps are unchanged, the relative expression of nog1 gene in Guichao 2 is obtained.

The relative expression of the nog1 gene in Guichao 2 is taken as 1 to count the relative expression of the nog1 gene in other rice plants. The results showed that the relative expression of nog1 gene in each of three RNAi interference plants of T₀ generation is significantly reduced as compared with Guichao 2.

The above results show that all of RNAi-1-T₀, RNAi-2-T₀ and RNAi-3-T₀ are RNAi interference plants of T₀ generation.

V. Obtainment and Real-Time Quantitative PCR Detection of Homozygous RNAi Interference Plants of T₂ Generation

The RNAi-1-T₀ to RNAi-3-T₀ are self-crossed for two consecutive generations to obtain homozygous RNAi interference plants of T₂ generation, and designated as RNAi-1 to RNAi-3, respectively.

Real-time quantitative PCR detection is performed on RNAi-1 to RNAi-3 and Guichao 2 respectively according to the method of step 4,

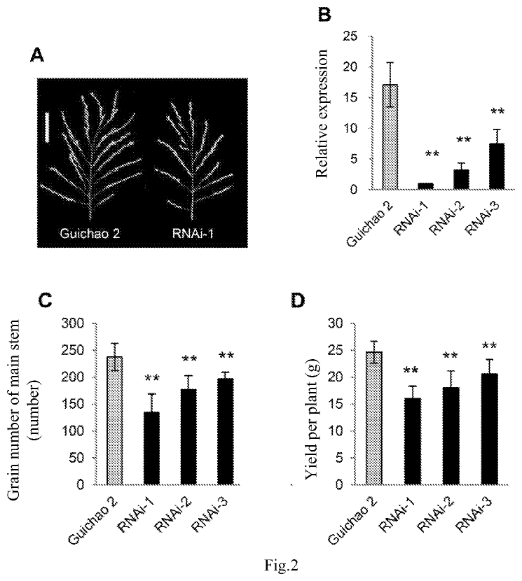

Some experimental results are shown in FIG. 2B (** indicates P<0.01, the difference is extremely significant). The results showed that the relative expression of the nog1 gene in each of RNAi-1 to RNAi-3 is significantly reduced as compared with Guichao 2.

VI. Phenotypic Identification of Homozygous RNAi Interference Plants of T₂ Generation

The seeds of the rice to be tested (Guichao 2, RNAi-1, RNAi-2 or RNAi-3) are planted in pots containing nutrient soil and vermiculite (the volume ratio of nutrient soil to vermiculite is 1:1) respectively, and cultured at 25° C. with alternative light, and the morphology of grains of the main stem, grain number of the main stem, the average grain number and the yield per plant of the rice to be tested are compared and counted during the growth and development. The experiment is repeated three times, with 30 plants repeated each time.

Some experimental results are shown in A, C and D of FIG. 2 (A is the morphology of grains of the main stem, bar=5 cm; C is the grain number of the main stem; D is the yield per plant; and ** indicates P<0.01, the difference is extremely significant). The results showed that grain number of the main stem, average grain number and yield per plant of each of RNAi-1, RNAi-2 and RNAi-3 are significantly reduced as compared with Guichao 2.

### Example 3. Obtainment and Phenotypic Identification of Homozygous Complementation Line of T₂ Generation

I. Construction of a Recombinant Plasmid pCAMBIA1300-NOG1

The steps of construction of the recombinant plasmid pCAMBIA1300-NOG1 are as follows:

1. PCR amplification is performed using two-week seedlings of Guichao 2 as experimental materials to extract genomic DNA and using it as a template, and using 860HBF: 5′-GAAGATCTCATCTGATGCCTCATACTGA-3′ (the recognition site of restriction endonuclease BglII is underlined) and 860HBR: 5′-CCGACGCGTCATGCTTAGGCTGTTGAT-3′ (the recognition site of restriction endonuclease MluI is underlined) as the primers to obtain a PCR amplification product of about 7 kb.

2. The PCR amplification product is digested with restriction endonucleases BglII and MluI, and a digested product is recovered.

3. The modified plant expression vector pCAMBIA1300 is digested with restriction endonucleases BglII and MluI, and a vector backbone of about 9 kb is recovered.

4. The digested product is ligated to the vector backbone to obtain a recombinant plasmid pCAMBIA1300-NOG1.

According to the sequencing results, the recombinant plasmid pCAMBIA1300-NOG1 is structurally described as follows: the modified plant expression vector pCAMBIA1300 in which the small DNA fragment between the recognition sequence of restriction endonuclease BglII and the recognition sequence of restriction endonuclease MluI is replaced with a DNA molecule with the nucleotide sequence as shown in Sequence 3 in the Sequence Listing.

II. Obtainment of Recombinant Agrobacterium

The recombinant plasmid pCAMBIA1300-NOG1 is introduced into Agrobacterium tumefaciens EHA105 to obtain a recombinant Agrobacterium EHA105/pCAMBIA1300-NOG1.

III. Obtainment of complemented plants of T₀ generation

The recombinant Agrobacterium EHA105/pCAMBIA1300-NOG1 is transformed into SIL176 using the method of Hiei et. al. (Hiei Y, Ohta S, Komari T & Kumashiro T. Efficient transformation of rice (Oryza sativa L.) mediated by Agrobacterium and sequence analysis of the boundaries of the T-DNA. Plant J. 1994, 6:271-282) to obtain complementation plants of T₀ generation.

IV. Real-Time Quantitative PCR Detection of Complemented Plants of T₀ Generation

Three complemented plants of T₀ generation (named as CTP-1-T₀ to CTP-3-T₀ respectively) are randomly selected for real-time quantitative PCR detection, the specific steps are as follows:

1. The total RNA is first extracted with TRIZOL reagent using two-week seedlings of three complemented plants of T₀ generation as experimental materials respectively, and then reverse transcribing is performed using SuperScriptII reverse transcriptase to obtain cDNA of each of complemented plants of T₀ generation. The amount of DNA in the cDNA of each of three complemented plants of T₀ generation is about 200 ng/μL.

2. The relative expression of nog1 gene in three complemented plants of T₀ generation is respectively detected using RT-qPCR technique (using UBI gene as internal reference gene).

The primers for detecting nog1 gene are forward primer 1: 5′-TCCGACTTACAATGAACAC-3′ and reverse primer 1: 5′-GGTAGCAGGACTCCACTT-3′. The primers for detecting UBI gene are forward primer 2: 5′-CTGTCAACTGCCGCAAGAAG-3′ and reverse primer 2: 5′-GGCGAGTGACGCTCTAGTTC-3′.

According to the above method, the complemented plants of T₀ generation is replaced with SIL176, and the other steps are unchanged, the relative expression of nog1 gene in SIL176 is obtained.

The relative expression of the nog1 gene in SIL176 is taken as 1 to count the relative expression of the nog1 gene in other rice plants. The results showed that the relative expression of nog1 gene in each of the three complemented plants of T₀ generation is significantly increased as compared with SIL176.

The above results showed that all of CTP-1-T₀, CTP-2-T₀ and CTP-3-T₀ are complemented transgenic rice of T₀ generation.

V. Obtainment and real-time quantitative PCR detection of homozygous complemented Lines of T₂ Generation

The CTP-1-T₀ to CTP-3-T₀ are self-crossed for two consecutive generations to obtain homozygous complemented plants of T₂ generation, which are designated as CTP-1 to CTP-3, respectively.

Real-time quantitative PCR detection is performed on CTP-1 to CTP-3 and SIL176 respectively according to the method of step 4.

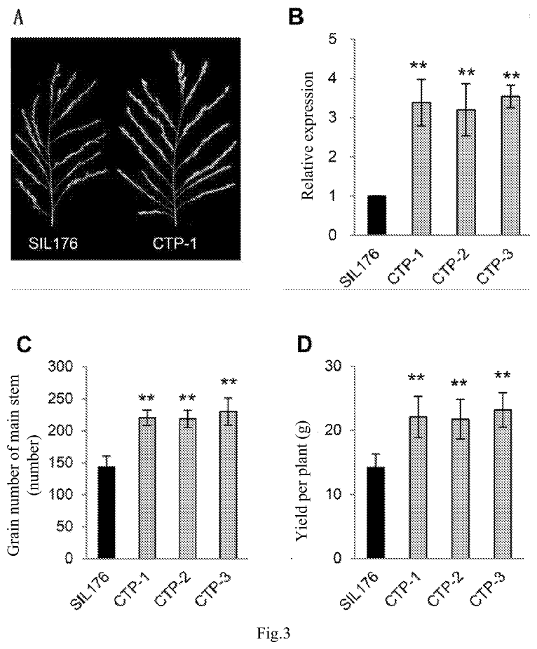

Some experimental results are shown in FIG. 3B (** indicates P<0.01, the difference is extremely significant). The results showed that the relative expression of the nog1 gene in each of CTP-1 to CTP-3 is significantly increased as compared with SIL176.

VI. Phenotypic Identification of Homozygous Complemented Plants of T₂ Generation

The seeds of the rice to be tested (SIL176, CTP-1, CTP-2 or CTP-3) are planted in pots containing nutrient soil and vermiculite (the volume ratio of nutrient soil to vermiculite is 1:1) respectively, and cultured at 25° C. with alternative light, and the morphology of the grains of main stem, grain number of the main stem, the average grain number and the yield per plant of the rice to be tested are compared and counted during the growth and development. The experiment is repeated three times, with 30 plants repeated each time.

Some experimental results are shown in A, C and D of FIG. 3 (A is the morphology of the grains of main stem, bar=5 cm; C is grain number of the main stem; D is the yield per plant; and ** indicates P<0.01, the difference is extremely significant). The results showed that grain number of the main stem, average grain number and yield per plant of CTP-1, CTP-2 and CTP-3 are significantly increased as compared with SIL176.

A substance which inhibits the expression of the protein nog1 is introduced into a starting rice (such as Guichao 2) to obtain a transgenic rice having a reduced yield per plant and/or a reduced grain number of the main stem. A nucleic acid molecule encoding protein nog1 is introduced into a starting rice (such as SIL176) to obtain a transgenic rice having an increased yield per plant and/or an increased grain number of the main stem and/or an increased average grain number. Therefore, protein nog1 can regulate rice yield and grain number and has an important application value.

1. A method of regulating yield and/or grain number of a plant, comprising;
introducing a nucleic acid molecule comprising a nucleotide sequence encoding a nog1 protein, wherein the protein nog1 is selected from the group consisting of a1), a2), a3) and a4):
a1) a protein with an amino acid sequence shown in SEQ ID NO: 2;
a2) a fusion protein obtained by linking a tag to the N-terminus or/and C-terminus of the protein shown in SEQ ID NO: 2;
a3) a protein which relates to plant yield and/or grain number obtained by substitution and/or deletion and/or addition of one or more amino acid residues in the amino acid sequence shown in SEQ ID NO: 2; and
a4) a protein having 80% or more identity with the amino acid sequence defined in a1);
screening the transgenic plant for the nucleic acid molecule; and
breeding the transgenic plant, wherein the transgenic plant exhibits increased yield and/or increased grain number compared to a non-transgenic plant.

2. (canceled)

3. The method according to claim 1, wherein the nucleotide sequence is a DNA molecule selected from the group consisting of b1), b2), b3) and b4) below:
b1) a DNA molecule with an encoding region as shown SEQ ID NO: 1;
b2) a DNA molecule with a nucleotide sequence as shown in SEQ ID NO: 1;
b3) a DNA molecule having 75% or more identity with the nucleotide sequence defined in b1) or b2) and encoding the protein nog1 of claim 1; and
b4) a DNA molecule hybridizing to the nucleotide sequence defined in b1) or b2) under stringent conditions and encoding the protein nog1 of claim 1.

4. The method according to claim 1, wherein the regulation of plant yield is a regulation of yield per plant.

5. The method according to claim 1, wherein the regulating grain number of the plant is a regulation of grain number of main stem and/or average grain number of the plant.

6. The use according to claim 1, wherein the plant is selected from the group consisting of c1) dicotyledon; c2) monocotyledon; c3) gramineous plant; c4) rice; c5) indica rice; c6) rice variety Guichao 2; and c7) Dongxiang common wild rice introgression line SIL176.

7. A method A for producing a transgenic plant A or a method B for producing a transgenic plant B, wherein:
the method A for producing the transgenic plant A comprises introducing a the nucleotide sequence encoding the protein nog1 of claim 1 into a recipient plant A to obtain the transgenic plant A; and the transgenic plant A has an increased yield and/or an increased grain number compared to the recipient plant A; and
the method B for producing the transgenic plant B comprises introducing a substance which inhibits the expression of the protein nog1 of claim 1 into a recipient plant B to obtain the transgenic plant B; and the transgenic plant B has a reduced yield and/or a reduced grain number compared to the recipient plant B.

8. The method A for producing the transgenic plant A of claim 7, wherein the introducing the nucleotide sequence encoding the protein nog1 into the recipient plant A is achieved by introducing a recombinant vector A into the recipient plant A; and the recombinant vector A is a recombinant plasmid obtained by inserting the nucleotide sequence encoding the protein nog1 into an expression vector.

9. The method B for producing the transgenic plant B of claim 7, wherein the substance which inhibits the expression of the protein nog1 is a DNA molecule, an expression cassette containing the DNA molecule, or a recombinant plasmid containing the DNA molecule;
the DNA molecule comprises a sense fragment, an antisense fragment and a spacer fragment located therebetween;
the sense fragment is a reverse complement of the DNA molecule shown at positions 155 to 522 from the 5′ end of SEQ ID NO: 1; and
the antisense fragment is a DNA molecule shown at positions 161 to 522 from the 5′ end of SEQ ID NO: 1.

10. A method C for cultivating a transgenic plant C, comprising introducing a substance which increases the expression and/or activity of the protein nog1 of claim 1 into a recipient plant C to obtain the transgenic plant C; and the transgenic plant C has an increased yield and/or an increased grain number compared to the recipient plant C.

11. (canceled)

12. The method of claim 7, wherein the plant is selected from the group consisting of f1) monocotyledon; f2) dicotyledon; f3) gramineous plant; and f4) rice.

13. The method of claim 7, wherein the yield is the yield per plant.

14. The method of claim 7, wherein the grain number is grain number of the main stem and/or average grain number.

15. (canceled)

16. The method of claim 1, wherein the nucleic acid molecule comprises a recombinant expression vector.

17. The method of claim 1, wherein the recombinant expression vector comprises SEQ ID NO.:1 or SEQ ID NO.:3.

18. The method of claim 10, wherein the plant is selected from the group consisting of f1) monocotyledon; f2) dicotyledon; f3) gramineous plant; and f4) rice.

19. The method of claim 10, wherein the yield is the yield per plant.

20. The method of claim 10, wherein the grain number is grain number of the main stem and/or average grain number.


SEQUENCE LISTING (3 sequences)

SEQ ID NO: 1 - DNA, 1170 bp, Artificial Sequence
  synthesized nucleotide
atggcggagccggagcagcagcagcagcaggcgaatcccgacgaggtggtgctcgggcaggagacaggcggcgcgagggtggcgatcctcaaccggccgcgccagctgaacgtcatctccgatagagtggtgtatctcctcgcccagttcttggagagctgggagaaagatgaggatgccaagctggtcatcttcaagggggctggacgtgcattttccgctggtggggatctaaagatgttctatgaaggaaaatcagatgactcctgtctcgaggttgtttacaggatgtattggctttgctaccatatccacacgtataagaaaaccgcggtggctcttgttaatggacttgtcatgggtggtggtgcagccatggttgctccactgaagtttgcagttgtcacagagaaaacagtcttcgcaacccctgaggctagtgttggattacacacagactgcagcttttcttatatccattctagactccctggatatttaggggagtacctggctttgaccggtgcaaggttgaatgcaaaggaaatgattgctgccggtcttgctactcattttgttccttctgaaaaattggaagaacttgaaaaatgcctgctgaatttaaacacaggagatgagtctgctgttcgagctgctattgaagagttctcaactgatgttcaacctgatgaagatagtattttaaacaagctcccaactatcaacaaatgtttctctgctgagactatcgaggacatcataaaagcttttgaatcagaagggagcattgatggaaaccaatggatcgctacagtactgaagggcatgcgaagatcatctcctacttcactgaagatgactcttcgatcgatcagagaaggtcggaagcagagcctgccggaatgtttgaagaaggaattccgacttacaatgaacactctccgatctgtagttactggcgatgtctatgagggaattagagctctcagcatcgacaaagacaatgcccctaagtggagtcctgctacccttgaggaggtcaagaacgaggacatcgaccgtcttttcgaaccattcagttcagaaaaggagctccaagtcccatctgacgattccaacagatggagtggcaaatttgagcacacagtctatggcagaacttcagagtaa

SEQ ID NO: 2 - protein, 389 aa, Artificial Sequence
  synthesized polypeptide
MAEPEQQQQQANPDEVVLGQETGGARVAILNRPRQLNVISDRVVYLLAQFLESWEKDEDAKLVIFKGAGRAFSAGGDLKMFYEGKSDDSCLEVVYRMYWLCYHIHTYKKTAVALVNGLVMGGGAAMVAPLKFAVVTEKTVFATPEASVGLHTDCSFSYIHSRLPGYLGEYLALTGARLNAKEMIAAGLATHFVPSEKLEELEKCLLNLNTGDESAVRAAIEEFSTDVQPDEDSILNKLPTINKCFSAETIEDIIKAFESEGSIDGNQWIATVLKGMRRSSPTSLKMTLRSIREGRKQSLPECLKKEFRLTMNTLRSVVTGDVYEGIRALSIDKDNAPKWSPATLEEVKNEDIDRLFEPFSSEKELQVPSDDSNRWSGKFEHTVYGRTSE

SEQ ID NO: 3 - DNA, 6921 bp, Artificial Sequence
  synthesized nucleotide
catctgatgcctcatactgacaattcccagtttgcgctgatattggtttactgaaatattataacgttgctattttgctcactgcttatggtagcccaattgaataatccgtaaaatcatggtctgattgaactgtgtttcaattttcttgctgtatatgctaatcttagtgacttgcatcaattgggtgttatgtgcttattacgtaattctgtaaaacgaaaaatacaagttaattcttcatgattgttttttgtttcatgctttcatatttcattggttatttgtttggcacaaccgcacaaagctttagctatctattttggctgatattgggtatgcatcattttactgatagggatattgattgatgctggataaaaatgtttttcttgcatcacattcaaacttttgagtagttaggatggcaactttgatggcaactttgccgaggttaccaacagttaatttggttccctttttttccctgttgcctatatctgattaactcttgggtgctctcttttttccataaacctagtggtttattggataagcatcctagctaacatcagctgctaaaataaagtagttttactttttaagttctcttactagtcgcattgtgaggttttgttaggcaactttgccgaggttaccaacagttaatttggttacctttttttccctgttgccaatatctgattagctgttaggtgctattctttccataaacctagtggtttaatggatgagcatcttagctaacatcagctgctaaaataaataacgatgtacttttaagttctgatcattctttcgttttgttttcataatctaattcaatgttctgtgactccataaatcatcttttttacttgggctttatatctcttcgtaatgcaagacggtatgcaaattttggtcatttcaattatgtacaaactcatcccagttgcatttgctacctgtgaagttgttaggcaactttgccgaggttaccaagacatgtttggtttcctttttttccctgttgcctatatcattagttgcttggttgcttgaactccattgacctatttggttttttgggtatgcatccatgccaacatataatattttccatcattctttttacacttagcactgcccaaatgttggtttggttcgtggactgagattctcgggaggcttttatgttttttgatcgaatggttcgtggtttctttgctggtttgtggttattttatcttcttggagtatatgtttgtcgagataattgtggatgtaaatgcagtcgtaaagctcttaaacgaactctaccaacagctcttattccgaaaaagtagctaaactgaaggatgaagaactctggttgggcggccgagcacctgattatcggccactgccttacgagtactttccgtctgtgaggtcgactcgacatcaacccttggaattccgtctgtgtggtacacaggatttgttatttagtaattgtaaccattcaaattatctactgctattcatttcaagttttcaacttacctagaagtgtttttagtcgctcgtcaatcgtcatgataacgaatgctcatacaacaaactgaaccagacgatacggacgaaactgcatgaagctagaatagctgtgtgttctagcagtgttgtctcgtagtaatactgtacgtaccagatcgtccagtacattcaaatttttcaccaagtcaagaactgagttctggaatcgttaagtgaagaactgagttctcgaatcgtgtgtgatttgtatagttccgagattagtactccctccgattaaggttttaatacgttgttagtaaaaaaaaggttttaatacgttttgattttagtcaaagttaaagttaaactgttttaagtttgactaaatttatagacaaatataataatatttataatactaaattagttttatcaaatcaataattaaatatattttcataataaatttattaatggaatatattttcataataaattgtttggggttaaaaatgttactatttttttatacaaacttggttaaacttaaatcagtttgactttgactaaactcaaaatgttttataatctaaaacagagggagtagtaatatatactctctctgtctcattttaagtgcaactatgattttccgtatccaacgttgattttccgttttatttgaaaattttttataattagtatttttattgttatgagatgataaaatataaatagtaatttatgcgtgacttatattttagcttttttttcaaataagacgaaaaatcgtagtcacacttaaaatggaaatggggcggcggagggaggactagtccactgataagtgataacgcatcattcaaaatgatcccgaagtgaaaaccgataaattctcaaagaggtgtgaattagaagtcaaactgcatcgacccagctgtccatggtctttttccgttcgccctcgctttcttcccgcaccaaccccgcctaccgctccaccactactactacaccatcccctccaccggctccccctcaccaaacttccctccacgcctgcttcgcccaactcgttctcttggagcacctaactcgagctgagctcccttcccgcggaattcgggtccctccctgatggcggagccggagcagcagcagcagcaggcgaatcccgacgaggtaccgatcagccccacgcgaacgaggccattttcttctttcttgtagtatgtggtgtggtgagagtgagaacgcgcggcgccgtttctttgtgcaggtggtgctcgggcaggagacaggcggcgcgagggtggcgatcctcaaccggccgcgccagctgaacgtcatctccgatagagtggtgcgatttctttcgcttcgtgcattttccgatcttatgccggaagcagcagctggaagctgtaattggctgggccgtgctcggtttctggttgcaggtgtatctcctcgcccagttcttggagagctgggagaaagatgaggatgccaagctggtcatcttcaaggtgcgcgcactgctgccctcttagcaccctgcattgataaagctctagggcagaacataactgattataattcagacaaggataggttttagcctagtgatgtcatgcgtgatttgataccaggagacagctcaaacaaatcatttaccttactctagtttgtttcttcaaagtccttgcaatgcatggagcacttggctagtagtacaaactatgtagagaaacgtgttgcttgtttaaggattcggtaaggtgtgcctggtcaacatttgaactttactcagtagttagtactgatacataaggtaacgttttgatatggataagtttggcaataagacagcgatgaaatgaggcgtatcatgtcacaaatgaaactttttgttatgactttgtatgatacccatcgattggcccaagcatatgaatgtatggtctgctgatgaacagggggctggacgtgcattttccgctggtggggatctaaagatgttctatgaaggaaaatcaggtatgaatatgatatgaatgtttcagtgtagcagtggtaccacatcacatgaccttactgtataatttgatttccatccccaagactcacatttgcgcacacttgatggcatttaaacctattaacagaactcctttgcagatgactcctgtctcgaggttgtttacaggatgtattggctttgctaccatatccacacgtataagaaaaccgcggtgatgccctcttttatgtttgtgggaggactgttttctaacttgggggcgaattctgtactcaaattctatggttttatttcaattcatgcaggtggctcttgttaatggacttgtcatgggtggtggtgcagccatggttgctccactgaagtttgcagttgtcacagagaaaacagtatgcaacactagtccatagtccagttcttttggaggctttttttatgccatattgtgttatatgtttgtttgttggtttgctgtatttatctgtaaaagtgtaaatcacccctctacatgtcactatctcttcaattcagctagtattattattttggcaaaattggttatatagcatcgaaagttcacgtttttggtatgaaaaaaatagaaaggagagaacactcctattcttgaaatggacccatttggtatcatgattactctgtcttgggagtttcgcggtgatgttttttgtgtgtttaggtcttcgcaacccctgaggctagtgttggattacacacagactgcagcttttcttatatccattctagactccctggatatttaggtaagtactgaattgcattttccattcagaggtcaaaccatcaccttactacaggcgtattgtctcttgagatgtaatgagaaatctgatagtgacttgtgtgacaaattaaacatatcttaggacaatatggttctgtaatgtatcacttgacatagatatttgtgccaatatgcttattctctttgaataattcttaggggagtacctggctttgaccggtgcaaggttgaatgcaaaggaaatgattgctgccggtcttgctactcattttgttccttctgaagtaaggcattcagcatttaccattcatattcacgatggttccggtgcatgatttcctcgattgttctacatgtagctcgacaaatcaaaatgtttgttgatatattgtctttctgtggttggactaaccaaaaatgattataatgtgaatggcagaaattggaagaacttgaaaaatgcctgctgaatttaaacacaggagatgagtctgctgttcgagctgctattgaagagttctcaactgatgttcaacctgatgaagatagtattttaaacaagttaagaactgatgtccttttccaaatgtaacttgctgcgcatgcatttattattctattattgaaataaattgacactggaatgacaatcttacttcgataggctcccaactatcaacaaatgtttctctgctgagactatcgaggacatcataaaagcttttgtaaggattctttcaattacttgcttgaaaagtgttgagttcctcagactatagaatattcatcttggtttcatgctacaaaataatctaaacaacaataagtggacagtattcatcttatgcccaaaatatatcatcgtcaaccaatagcttctggaaaaactagacacagtacactcaccaatcaccaccagtcttcactgtcctataacaaggcaatcaactttttcctgcaaatcataagcagcgtcttttgtttttgttgttgttgttgtttgtaggaatcagaagggagcattgatggaaaccaatggatcgctacagtactgaagggcatgcgaagatcatctcctacttcactgaagatgactcttcgatcggtacaaactctgacatcgtttctaatgctgatgattttgtctcgtcttaataatatttgtgcttcttatgtatggtgaatgcttaaaagctatagtagctgtccgcttctccttggttaacaatttatttatatgttgtgcagtgtactaatttttgacattttgttatgtattgtggtgcatttacagctcctttttgcacaaggcaagaatgtattcttaatattaatggttgttttaattcttcagatcagagaaggtcggaagcagagcctgccggaatgtttgaagaaggaattccgacttacaatgaacactctccgatctgtagttactggcgatgtctatgaggtactgtgatttgccactatatgaataagtaacggtgattcttgatacaggcaagactaagatgtcattcatcataatatttgcacctaaaacttggcagggaattagagctctcagcatcgacaaagacaatgcccctaaggttctctcatacacacttttaaacatgtctagaaatttttatttttcatactgtttcttaatttttgttgcgatacagtggagtcctgctacccttgaggtcaagaacgaggacatcgaccgtctttcgaaccattcagttcagaaaaggagctccaagtcccatctgacgattccaacaggtgaacaattgctccaaaatccatttcttttctctgcccttcacacaaacaacagtgtatcctaatcatctgcactatttccaattttgacatggacaatgcacagatggagtggcaaatttgagcacacagtctatggcagaacttcagagtaattggcagcagaaatagatgttcatcatcctcacaagcatgttttaagtactcgtaaccccatgtattctcatacttccctgtataacattgtaacacaaaaaattgaaaattacatgacgcacgaagcaataaagcatctcaaatgttttgttacatcctgaagccaatgaataagaacttttccggcgtgtgatctctgtaaagagaattccaacctaagggttaaagtctagggagtgaatgtatgtccaagagagaccagtcatgtagcgacttgagagccctatgagcagttgggctccacaggatactctaactccaagtagtattgtttgtacttggtagttttcttctgagtgggggctttcctcactgacaaaattctcctcacagcagcttcttggtctttcgggaactccaccgccatcttcttcaggttcgttgcttgagttagcagaaagataaagagttccttctggtaccgttgaggcctgaatccaagaaacttgagctgctccaaactcttagttaggcatgataactttgaaagcctctcctcatgatcagcaaaatttggcaatgcagtgtcgtcttttgcatcaacagcctaagcatgatcaacagcctaagcatg